(19) United States
(12) Patent Application Publication
(10) Pub. No.: US 2014/0023715 A1
(43) Pub. Date: Jan. 23, 2014

(54) POLYMER CONJUGATES TARGETING CELLS AND METHODS RELATED THERETO

(71) Applicants: Lily Yang, Atlanta, GA (US); Gee Young Lee, Atlanta, GA (US); Malgorzata Lipowaska, Decatur, GA (US); Hui Mao, Johns Creek, GA (US)

(72) Inventors: Lily Yang, Atlanta, GA (US); Gee Young Lee, Atlanta, GA (US); Malgorzata Lipowaska, Decatur, GA (US); Hui Mao, Johns Creek, GA (US)

(73) Assignee: EMORY UNIVERSITY, Atlanta, GA (US)

(21) Appl. No.: 14/005,499

(22) PCT Filed: Mar. 30, 2012
(86) PCT No.: PCT/US12/31341

§ 371 Date: Oct. 4, 2013

Related U.S. Application Data
(60) Provisional application No. 61/471,317, filed on Apr. 4, 2011.
(60) Provisional application No. 61/469,382, filed on Mar. 30, 2011.

(51) Int. Cl. A61K 47/48

(52) U.S. Cl. CPC A61K 47/48246
USPC 424/491; 514/21.9; 424/181.1; 514/19.3; 514/19.4; 514/19.5

(57) ABSTRACT

This disclosure relates to polymer coated particles targeting cancer cells and methods related thereto. In certain embodiments, the disclosure relates to nanoparticles coated with amphiphilic polymers conjugated with molecules useful for targeting tumors, monitoring the location of the nanoparticles administered to a subject by MRI, and viewing the presence of the nanoparticles during optical image-guided surgery.

### CROSS REFERENCE TO RELATED APPLICATIONS

This application claims priority to U.S. Provisional Application No. 61/469,382 filed Mar. 30, 2011 and U.S. Provisional Application No. 61/471,317 filed Apr. 4, 2011, both hereby incorporated by reference in their entirety.

### ACKNOWLEDGEMENTS

This invention was made with government support under Grant Nos. UO1CA1151810, U54CA119338. P50CA128301. P50CA28613 and R01CA133722 awarded by the National Institutes of Health. The government has certain rights in the invention.

### BACKGROUND

Many people die of cancer each year. Chemotherapy and radiotherapy are options for these patients. However, the treatment of certain cancers with chemotherapy and radiotherapy is not satisfactory, e.g., pancreatic cancers are highly resistant to chemotherapy. It is common to remove cancerous tumors surgically. Operatively removing tumors often results in recurrence locally and at distal locations. For example, triple negative breast cancers have a high incidence of tumor recurrence despite surgical interventions. Visualizing small growths can be extremely difficult, and locating where a tumor starts and stops during surgery can be challenging. Pre-tumor forming cancerous stem cells may exist in a tissue creating a formidable challenge to solely relying on a surgical intervention as a therapeutic strategy. Thus, there is a need to identify improved methods of treating cancer.

Theranostics are therapeutics with physical properties that allows one to image molecular accumulation of the therapeutic vehicles in vivo. Yang et al., WO/2007/018647, disclose binding and internalization of tumor targeted-iron oxide particles using MRI. See also Yang et al., J. Biomed. Nanotechnol., 2008, 4, 439-449. Lammers et al., Biomaterials, 2009, 30(2):3466-3475, disclose the simultaneous delivery of doxorubicin and gemcitabine to tumors in vivo using polymeric drug carriers.

### SUMMARY

This disclosure relates to polymer coated particles targeting cells and methods related thereto. In certain embodiments, the disclosure relates to nanoparticles coated with amphiphilic polymers conjugated with molecules useful for targeting tumors, monitoring the location of the nanoparticles administered to a subject by MRI, and viewing the presence of the nanoparticles during optical image-guided surgery.

In certain embodiments, the disclosure relates to particles comprising a core coated with a polymer, wherein the polymer is conjugated to a targeting moiety, a lysosomally degradable moiety, and a therapeutic agent such as gemcitabine, doxorubicin, cytosine arabinoside, mitomycin, or any therapeutic agent with that an amine side group. In certain embodiments, the therapeutic agent is cisplatin. In certain embodiments, the particle is a metal nanoparticle or metal oxide nanoparticle, such as an iron oxide nanoparticle or elemental iron core nanoparticle with an oxide coat, or a quantum dot, e.g., those with a diameter of between about 5 to 200 nm or 10 to 100 nm. In certain embodiments, the lysosomally degradable moiety is the polypeptide GFLG (SEQ ID NO: 1) linked to the therapeutic agent.

In certain embodiments, the disclosure relates to compositions comprising a polymer conjugated to a targeting moiety, lysosomally degradable moiety, and a therapeutic agent which are described herein.

In one example, the lysosomally degradable moiety linked to the therapeutic agent is of the formula:

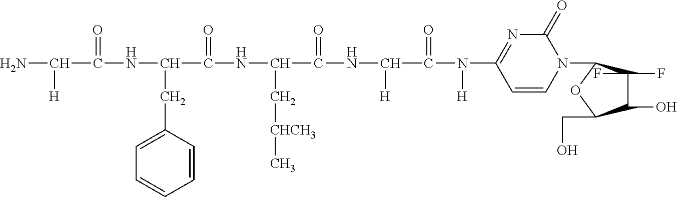

or salts or derivatives thereof optionally substituted with one or more substituents.

In certain embodiments, the polymer is an amphiphilic polymer comprising a hydrophobic section further comprising a hydrophobic therapeutic agent.

In certain embodiments, the targeting moiety binds uPAR, a EGFR, or HER-2, PMSA, folate receptor, transferrin receptor, MUC-1, integrin alpha vbeta, cell surface nucleolin, CTLA-4, or VEGFR. In certain embodiments, the targeting moiety is an antibody or antibody mimetic, or aptamer of a natural ligand thereof such as the amino-terminal fragment of uPA, EGF, or folic acid.

In certain embodiments, the particle further comprises a (3,3-dimethyl-indol-1-ium-1-yl)-N-alkylsulfonate dye or salt thereof such as one of the formula:

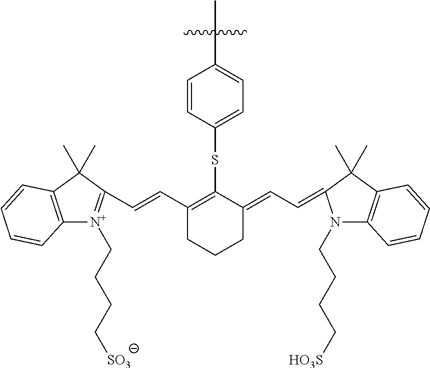

or salts or derivatives thereof optionally substituted with one or more substituents.

In certain embodiments, the disclosure relates to pharmaceutical compositions comprising particles, conjugates, and compounds disclosed herein and a pharmaceutically acceptable excipient and optionally another anti-cancer agent. The pharmaceutical composition may be in the form of a buffered saline solution, gel, pill, capsule, or tablet. In certain embodiments, the pharmaceutical compositions may be administered in combination with other anti-cancer agents.

In certain embodiments, the disclosure relates to methods of treating or preventing a disease or condition comprising administering an effective amount of a pharmaceutical composition disclosed herein to a subject in need thereof. In certain embodiments, the disease is cancer that forms tumors. In certain embodiments, the cancer overexpresses a receptor of the targeting molecule in tumor cells, tumor endothelial cells, or tumor stromal fibroblasts compared to noncancerous tissue of an organ containing the cancerous tumor. In certain embodiments, the targeting molecule is an antibody or fragment, antibody mimetic, inhibitor, or aptamer targeting a protein or glycoprotein expressed on the surface of a cancerous cell. In certain embodiments, the cancer overexpress uPAR, EGFR, or HER-2. In certain embodiments, the cancer is selected from pancreatic cancer, breast cancer, prostate cancer, lung cancer, skin cancer, bladder cancer, brain cancer, colon cancer, rectal cancer, kidney cancer, endometrial cancer, and thyroid cancer.

In certain embodiments, the disclosure relates to conjugates comprising a lysosomally degradable moiety and a therapeutic agent. For example, the lysosomally degradable moiety may be the polypeptide GFLG (SEQ ID NO: 1) wherein the therapeutic agent is an anti-cancer agent. In certain embodiments, the therapeutic agent is gemcitabine.

In certain embodiments, the disclosure relates to compounds of the following formula:

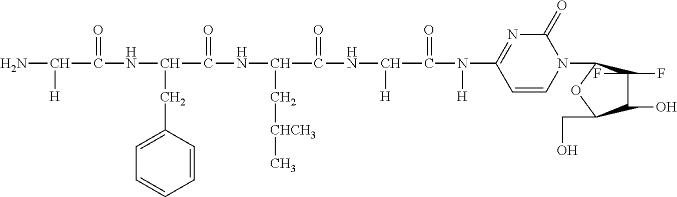

or salts or derivatives thereof optionally substituted with one or more substituents. In certain embodiments, the compound is conjugated to a targeting moiety such as an HER-2 or EGFR antibody, aptamer, or antibody mimetic, uPA or fragment thereof such as the amino-terminal fragment of uPA or single chain antibody to EGFR.

In certain embodiments, the disclosure relates to conjugates comprising a peptide or antibody and a dye of the formula:

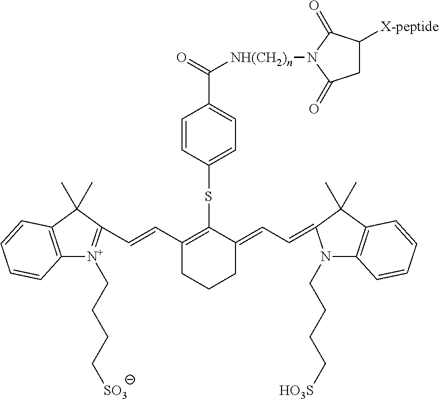

or salts or derivatives thereof optionally substituted with one or more substituents wherein X is S or NH and n is 2 to 22 or n is 4 to 22.

In certain embodiments, the disclosure relates to conjugates comprising a peptide or antibody and a dye of the formula:

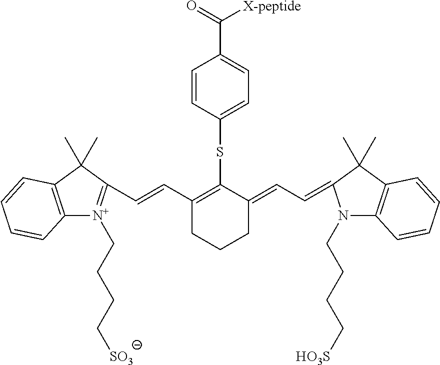

or salts or derivatives thereof optionally substituted with one or more substituents wherein X is S or NH.

In certain embodiments, the disclosure relates to dyes of the formula:

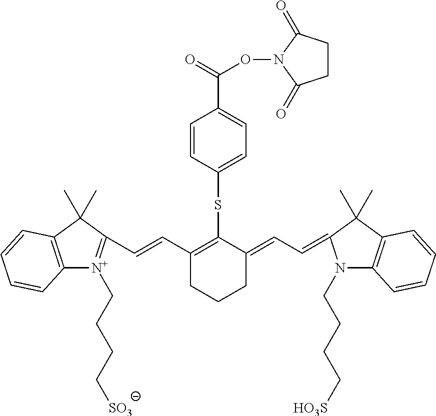

or salts or derivatives thereof optionally substituted with one or more substituents.

In certain embodiments, the disclosure relates to dyes of the formula:

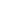

or salts or derivatives thereof optionally substituted with one or more substituents wherein n is 2 to 22 or n is 4 to 22.

In certain embodiments, the disclosure relates to compositions comprising a magnetic iron oxide nanoparticle comprising an amphiphilic polymer comprising a hydrophobic region and a hydrophilic region conjugated with a dye and a targeting moiety. In certain embodiments, the dye is a (3,3-dimethyl-indol-1-ium-1-yl)-N-alkylsulfonate or salt thereof. In certain embodiments, the targeting moiety is an amino terminal fragment of uPA or a single chain antibody to EGFR. In certain embodiments, the polymer is further conjugated to an anti-cancer agent linked to a lysosomally degradable linker wherein the lysosomally degradable linker is covalently attached to the polymer. In certain embodiments, the lysosomally degradable linker is the sequence GFLG (SEQ ID NO:1) wherein the anti-cancer agent is gemcitabine. In certain embodiments, the hydrophobic region comprises a hydrophobic anti-cancer agent such as doxorubicin, ABT-888 (veliparib), erlotinib, taxol or salt thereof.

In certain embodiments, particles disclosed herein are administered an effective amount to treat a subject diagnosed with cancer or a cancerous tumor. In certain embodiments, the particles disclosed herein are administered in combination with a second anti-cancer agent such as, but not limited to, bevacizumab, gefitinib, erlotinib, temazolamide, docetaxel, cis-platin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vincristine, vinblastine, vindesine, vinorelbine taxol, taxotere, etoposide, teniposide, amsacrine, topotecan, camptothecin, bortezomib, anegrilide, tamoxifen, toremifene, raloxifene, droloxifene, iodoxyfene fulvestrant, bicalutamide, flutamide, nilutamide, cyproterone, goserelin, leuprorelin, buserelin, megestrol, anastrozole, letrozole, vorazole, exemestane, finasteride, marimastat, trastuzumab, cetuximab, dasatinib, imatinib, combretastatin, thalidomide, and/or lenalidomide or combinations thereof.

In certain embodiments, the disclosure relates to methods comprising preoperatively administering a composition comprising particles disclosed herein and monitoring the location of the particles in the subject by detecting it by MRI (magnetic resonance imaging) in an area of the subject. In certain embodiments, the method further comprises the steps of operating on the subject in the area of detected particles, imaging dye identified tumors binding the targeting moiety, and surgically removing dye identified tumors or tissue.

In certain embodiments, the disclosure relates to methods comprising preoperatively administering cancer targeted nanoparticles conjugated to dyes disclosed herein to a subject, optically imaging a tumor that bind the nanoparticles intraopertively, and removing tumors targeted with the nanoparticles.

In certain embodiments, the particle is further conjugated to a nucleic acid capable of expressing siRNA.

### BRIEF DESCRIPTION OF THE FIGURES

FIG. 1 illustrates the preparation of ATF-Gem-IONPs. (a) Synthetic scheme of GFLG-Gem conjugates and (b) diagram of the conjugation of ATF peptide and GFLG-Gem conjugates to IONPs.

FIG. 2 shows data on gemcitabine release from Gem-IONPs (upper) and ATF-Gem IONPs (lower) NPs measured by HPLC after 24 h-incubation with 1 μM of Cathepsin B or without enzyme at pH 5.5 (left column) or pH 7.4 (right column).

FIG. 3 shows in vitro indicating targeting activity and cytotoxicity of and Gem-IONPs and ATF-Gem-IONPs. (a) Bright field images of MIA PaCa-2 cells incubated with IONPs (A, D), Gem-IONPs (B, E), and ATF-Gem-IONPs (C, F) at 10 nM (A, B, C) and 100 nM (D, E, F) for 4 h. Iron was detected by the Prussian blue staining and samples were magnified 40×. (b) Cell proliferation after 96 h-incubation with serial dilutions (0.01˜100 M) of gemcitabine (black bar), Gem-IONPs (dark gray bar), ATF-Gem-IONPs (light gray bar), as determined using the Crystal violet assay. Data are expressed as percentages of the untreated control. The values shown are means±S.D. for quadruple samples.

FIG. 4 shows in vivo data on anti-tumor activity. (a) Mean tumor weight (blue bar) of non-, gemcitabine-, Gem-IONP, and ATF-Gem-IONPs treated MIA PaCa-2 bearing mice and individual weight distribution in every group (various symbols). Mice were given by i.v. injection of gemcitabine-equivalent dose of 2 mg/kg five times at 3- or 4-day intervals. At the end of the experimental period, tumor tissues were isolated and weighed. Values represent mean±S.D. for 16 mice. *Statistically significant difference vs. control, p<0.0001. **Statistically significant difference vs. gemcitabine and Gem-10 groups, p<0.05. (b) Representative tumor images of each group after dissection.

FIG. 5 shows T2-Weighted MR images and bright field images of a mouse treated with Gem-IONPs and with ATF-Gem-IONPs (A) and comparison of Spin-Echo and UTE MR images from a mouse treated with ATF-Gem-IONPs (B). All MRI were taken at 48 h after final injection. Yellow dotted circles and arrows indicate the location of primary tumor lesions in MR and BF images, respectively, and blue arrows indicate the secondary tumor lesions due to metastasis. Iron distribution in tumor tissues obtained from Gem-IONPs and ATF-Gem-IONPs groups detected by Prussian blue staining (C).

FIG. 6 schematically illustrates the preparation of NIR830-NHS and NIR830-Maleimide.

FIG. 7 schematically illustrates the reaction of the dyes with ATF.

FIG. 8 shows data on comparing the of optical signal using different NIR dye-conjugated mouse amino-terminal fragment of uPA (ATF) peptides, which binds to uPAR, using Olympus OV-100 (Cy5.5, IRDye800, and NIR-830). Yellow and white arrows show mammary tumor; Blue arrows: liver; White numbers in the image are the NIR signal to body background ratios. Commercial dye Cy5.5 (Amersham) demonstrated a high body background in Balb/c mouse and the mammary tumor was not detectable. IRDye 800 (Licor) had a high non-specific signal in the liver. NIR-830 was superior to the other dyes.

FIG. 9 shows data on optical imaging of primary and metastatic tumors over a 28 day time course. Optical signal of NIR-830 dye lasts over 15 days after targeting to tumor site. Primary tumor was removed 15 days after imaging probe injection. By day 28, bioluminescence imaging detects luciferase (+) tumor cells in the low abdominal area. The peritoneal metastasis is still detectable 9 days after re-administration of NIR-830-ATF probes (arrows).

FIG. 10 shows optical imaging of orthotopic cancer models using NIR-830 dye-labeled Her-2 targeted ligands. Left panel is a human breast cancer xenograft model with a Her-2 affibody labeled with NIR-830. Right panel is NIR 830 labeled monoclonal Her-2 antibody 24 hours after injection.

FIG. 11 shows images from using NIR-830 labeled Her-2 affibody conjugated to iron oxide nanoparticles for detection of Her-2 human ovarian cancer.

FIG. 12 illustrates uPAR theranostic IONPs with NIR dye. Electron Microscope (EM) image of 10 nm uniform size IONPs and amphiphilic polymer linked with polyethylene glycol (PEG) trimmer. The preparation of NIR-830-ATF-IONP or NIR-830-ScFvEGFR-IONP are illustrated.

FIG. 13 shows data on the targeted therapy of orthotopic pancreatic tumors using NIR-830-dye-ATF-IONP-Dox.

FIG. 14 shows Kodak FX in vivo imaging of NIR-830-ATF-IONP-Dox. Left panel shows the location of the primary tumor but no visible tumor lesions in retroperitoneal cavity; however, Kodak FX imaging provides the location of a small metastatic lesion on the ureter.

FIG. 15 shows SpectroPen detection of NIR signals in the primary tumor and the peritoneal metastasis.

FIG. 16 illustrates certain embodiments of the disclosure. Targeting ligands labeled with a NIR 830 dye are conjugated to the carboxyl group using EDAC to generate dual MRI and optical imaging IONP. A hydrophobic chemotherapy drug (Dox) can be encapsulated into the hydrophobic space between the IONP core and amphiphilic polymer. GFLG-Gem may also be linked to the IONPs. Nucleic acids conjugated to the polymers are contemplated, e.g., siRNA expressing DNA cassette and oligonucleotide aptamers.

### DETAILED DISCUSSION

Targeted Nanoparticles Comprising an Anti-Cancer Agent with a Lysosomally Degradable Linker

To develop effective approach for pancreatic cancer therapy, a multifunctional theranostic NPs system has been engineered that combines imaging capability and receptor specificity of the NPs designed to overcome physical and intrinsic barriers that confer drug resistance in pancreatic cancer. The sequence GFLG (SEQ ID NO: 1) is a lysosomally sensitive tetrapeptide. GFLG is conjugated to an anticancer agent that is used for pancreatic cancer therapy. Gemcitabine is conjugated to the amine end of the peptide and the other end of the peptide is bound to carboxyl group on the amphiphilic copolymer coated on the magnetic iron oxide NPs so that gemcitabine is released after endocytosis by receptor-mediated internalization.

An amino terminal fragment (ATF) of uPA was further conjugated to the IONPs resulting in uPAR targeted-IONPs. uPAR is highly expressed in pancreatic tumor and stromal cells. Using an orthotopic human pancreatic cancer xenograft model, systemic delivery of uPAR-targeted gemcitabine-IONPs significantly reduced the growth of pancreatic tumors, and the efficiency of drug delivery and response to therapy could be monitored by MRI. This theranostic NPs is a promising drug delivery system for breaking the physical barrier of enriched tumor stroma in pancreatic cancer and an effective tool for the treatment for pancreatic cancer.

Gemcitabine can be controlled released out at the tumor, and its delivery status, such as accumulation of the drugs and tumor response to treatment can be monitored by MRI, a non-invasive imaging method. The NPs released gemcitabine in the presence of cathepsin B at pH 5.5 and retarded pancreatic tumor growth more than 50% compared to control and showed 1.5-times higher tumor inhibition effect than gemcitabine or non-targeted IONPs. Targeting ability of ATF-Gem-IONPs in the cells and in the tumor tissues was confirmed by Prussian blue staining and MRI.

### Targeted Therapy and Imaging of the Dye Labelled NIR-830 Theranostic

Targeted tumor growth inhibition and anti-angiogenesis effects occur after systemic delivery of mouse ATF-IONP-Dox in 4T1 mouse mammary tumor model. T2-weighted MRI is able to image IONP-mediated drug delivery and response. Targeted delivery of ATF-IONP-Dox was further confirmed by Prussian blue staining of resected primary tumor tissue sections as well as ICP-OES analysis of iron concentration in the tumor lysate. Importantly, preoperative treatment of the tumor-bearing mice with ATF-IONP-Dox significantly inhibited local recurrence and distant metastasis. Targeted delivery of Dox using IONPs reduced toxicity to the heart and liver.

Systemic delivery of ATF-Gem-IONPs into nude mice bearing orthotopic pancreatic cancers inhibits tumor growth and allows UTE MRI of tumors as a positive (bright) MRI signal in three areas inside the abdominal cavity that correspond well with the tumor lesions found in the mouse. NIR optical imaging may be performed using whole body imaging to determine target specificity and biodistribution using the Kodak Fx in vivo imaging system. SpectroPen optical imaging system, a pen-sized fiber-optic probe, may be used during surgery for wavelength-resolved raman and fluorescence measurements. See Mohs et al., Anal Chem, 2010, 82, 9058-9065, hereby incorporated by reference in its entirety. SpectroPen imaging has a high sensitivity and can detect as few as 5,000 of NIR-830-ATF labeled tumor cells injected under Balb/c mouse skin.

NIR-830-dye labeled ATF- or ScFvEGFR-IONPs specifically accumulated in the human breast cancer xengrafts after tail vein delivery and produced strong NIR signals that are detectable by both Kodak FX in vivo and SpectroPen imaging systems. T2-weighted MRI scan also shows targeted delivery of the IONPs in the tumor. SpectroPen detects high ratios of tumor signal to body background (>30). Tail vein injection of 20 pmol of NIR-830-ATF-IONPs per mouse, which only contains 400 ng of NIR-830 dye, is sufficient for targeted tumor imaging by SpectroPen.

### TERMS

The term “amphiphilic polymer” refers to a polymer composed of monomeric units with hydrophilic and hydrophobic groups, e.g., copolymer of monomers with hydrophilic groups and monomers with hydrophobic groups or homopolymers with monomer with hydrophilic groups and hydrophobic groups. Typical hydrophilic groups are hydroxy, amine, carboxylic acid and polyethylene glycol groups. Typical hydrophobic groups are long chain alkyl, carbocyclic, and aromatic groups

As used herein, “subject” refers to any animal, preferably a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, the term “derivative” refers to a structurally similar compound that retains sufficient functional attributes of the identified compound. The derivative may be structurally similar because it is lacking one or more atoms, substituted, a salt, in different hydration/oxidation states, or because one or more atoms within the molecule are switched, such as, but not limited to, replacing an oxygen atom with a sulfur atom or replacing an amino group with a hydroxyl group, replacing an aromatic CH with a nitrogen or sulfur. The derivative may be a prodrug. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, alkanoyl, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)2Ra, —OS(═O)2Ra and —S(═O)2ORa. Ra and Rb in this context may be the same or different and independently hydrogen, halogen hydroxyl, alkyl, alkoxy, alkanoyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

The term “optionally substituted,” as used herein, means that substitution is optional and therefore it is possible for the designated atom to be unsubstituted.

The terms “halogen” and “halo” refer to fluorine, chlorine, bromine, and iodine.

As used herein, “alkyl” means a noncyclic straight chain or branched, unsaturated or saturated hydrocarbon such as those containing from 1 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” group's are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The term “aryl” refers to aromatic homocyclic (i.e., hydrocarbon) mono-, bi- or tricyclic ring-containing groups preferably having 6 to 12 members such as phenyl, naphthyl and biphenyl. Phenyl is a preferred aryl group. The term “substituted aryl” refers to aryl groups substituted with one or more groups, preferably selected from alkyl, substituted alkyl, alkenyl (optionally substituted), aryl (optionally substituted), heterocyclo (optionally substituted), halo, hydroxy, alkoxy (optionally substituted), aryloxy (optionally substituted), alkanoyl (optionally substituted), aroyl, (optionally substituted), alkylester (optionally substituted), arylester (optionally substituted), cyano, nitro, amino, substituted amino, amido, lactam, urea, urethane, sulfonyl, and, the like, where optionally one or more pair of substituents together with the atoms to which they are bonded form a 3 to 7 member ring.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above with the indicated number of carbon atoms attached through an amino bridge. An example of an alkylamino is methylamino, (i.e., —NH—CH3).

“Alkanoyl” refers to an alkyl as defined above with the indicated number of carbon atoms attached through a carbonyl bride (i.e., —(C═O)alkyl).

### Polymer-Coated Particles

Within certain embodiment, the compositions and methods disclosed herein may be utilized with a variety of polymer coated particle such as, e.g., quantum dots (QDs), metal particles, gold, silver, iron, and iron-oxide nanoparticles (IONPs). IONPs are typically prepared with a mean particle diameter of 4-100 nm. IONPs may be prepared by aging a stoichiometric mixture of ferrous and ferric salts in aqueous media under basic conditions. Control over particle size (2-20 nm) and shape is provided by adjusting the pH, ionic strength and the concentration of the growth solution. The nanoparticles can be functionalized in situ using additives such as organic compounds (e.g. sodium citric) or polymers (e.g. dextran, polyvinyl alcohol). Other metals such as gold, cobalt, nickel, and manganese may be incorporated into the material.

High-temperature decomposition of Fe(CO)₅ in organic solvents is another way to prepare IONPs. Size (3-19 nm) can be varied using alternative temperatures. Flame spray pyrolysis yields a range of magnetite, maghemite and wustite (FeO) particles IONPs. Iron precursor such as Fe(CO)₅ and Fe(NO₃)₃ may be used. Flame spray pyrolysis can be used to produce different nanoparticles (TiO₂, ZrO₂, silica, etc.) as well as hybrid particles (e.g. silica-IONPs).

Hydroxyl groups on the IONP provide a place for synthetic attachment of different functional groups. A range of chemistries can be used to stabilize metal nanoparticles, exploiting electrostatic, hydrophobic, chelating and covalent interactions. Carboxylic acid groups can interact with the surface of IONPs by coordination processes. IONP synthesis in organic solvents is typically conducted in oleic acid. A polymer coating on the IONPs is preferred. Polymer attachment to the IONP surface by an initiator fixed to the surface of the IONPs and the polymer is grown from the surface. Alternatively, a functional, pre-formed polymer is grafted onto IONPs in situ. Copolymers with hydrophobic groups, carboxylic acid groups, polyethylene glycols, or amine groups are contemplated. Polymers with a hydrophilic block and a hydrophobic block are contemplated. See Yang et al., Clin Cancer Res, 2009 15:4722; Lin et al., Small, 2008, 4(3):334-341; Yu et al., Nanotechnology, 2006, 17:4483-4487; Park et al., J. Mater. Chem., 2009, 19, 6412-6417; Boyer et al. NPG Asia Mater., 2010, 2(1):23-30, Kim et al., Nanotechnology, 2011, 22, 155101; all hereby incorporated by reference in their entirety.

Conjugating molecules or polypeptides to the polymers can be accomplished using a variety of methods. Typically, primary amine containing compounds and proteins may be conjugated to the carboxylic acid groups on the polymer mediated by a coupling reagent such as EDAC. See Yang et al., Small, 2009, 5(2):235-43, hereby incorporated by reference in its entirety. Other coupling methods are contemplated, e.g., poly-histidine sequence may be recombinantly incorporated into a polypeptide sequence of the targeting moiety. A poly-histidine chelating agent may be coupled to the polymer surface, e.g., NTA-Ni. Mixing the histidine tagged polypeptide sequence attaches it to the polymer surface linked through the chelating agent. The avidin/streptavidin-biotin interactions may be used, e.g., biotin may be coupled to the polymer surface and streptavidin may be expressed as a chimera with the targeting moiety.

### Targeting Moieties

Urokinase plasminogen activator (uPA) is a serine protease that regulates multiple pathways involved in matrix degradation, cell motility, metastasis and angiogenesis. Interaction of the N-terminal growth factor domain of uPA with its cellular receptor (uPAR) results in the conversion of the plasminogen to a serine protease, which is a central regulator of the activation of other proteases including the matrix metalloproteinases (MMPs). Studies have shown that the uPA/uPAR complex controls the motility of both tumor and endothelial cells. In addition to its role in activation of the process for degradation of extracellular matrix, uPAR also activates α5β1 integrin and ERK signaling through interaction with EGFR and induces cell proliferation. Additionally, the uPA/uPAR complex can bind to the matrix protein, vitronectin, in association with transmembrane integrins, and activate intracellular signaling molecules such as the protein kinases, promoting cell adhesion, proliferation, and migration.

The cellular receptors for uPA (uPAR) are highly expressed in many human tumor cells, intratumoral fibroblasts and tumor endothelial cells. About 54% of ductal carcinoma in situ (DCIS) and 73% of lobular carcinoma tissues have over 50% of their cancer cells overexpressing uPAR. An elevated level of uPAR is associated with tumor aggressiveness, the presence of distant metastasis and poor prognosis. However, uPAR is undetectable in the majority of normal tissues or organs except for low levels expressed in macrophages, granulocytes, the uterus, thymus, kidney and spleen. Therefore, uPAR is an excellent molecular target for recruiting nanoparticles to breast tumor sites.

The uPAR-binding domain of uPA is located to the amino-terminal fragment (ATF) of uPA. Studies have shown that ATF is a potent uPA binding antagonist to its high affinity receptor (uPAR) at the surface of both tumor and endothelial cells. Systemic or local delivery of a non-catalytic amino-terminal fragment (ATF) of uPA (residues 1-135) using an adenoviral vector or conjugated peptides prevents the formation of the uPA/uPAR complex, thus inhibiting tumor growth and angiogenesis. Yang et al., Clin Cancer Res., 2009, 15(14):4722-32, hereby incorporated by reference in its entirety, discuss the preparation of targeted iron oxide nanoparticle using a recombinant peptide containing the amino-terminal fragment of urokinase-type plasminogen activator (uPA) conjugated to magnetic iron oxide nanoparticles amino-terminal fragment conjugated-iron oxide nanoparticle (ATF-IONP). This nanoparticle targets uPA receptor, which is overexpressed in breast cancer tissues.

The human epidermal growth factor receptor (EGFR) family includes EGFR (HER-1), EGFR-2 (HER-2), EGFR-3 (Her-3) and EGFR 4 (HER-4). The ligands that bind to EGFRs are divided into EGFR-like ligands such as EGF and TGF-α, and the heregulins. These ligands bind to EGFR monomers to promoter receptor dimerization and oligomerization that ultimately results in the activation of the EGFR signaling pathway. This EGFR signaling pathway plays a role in the regulation of cell proliferation, survival and differentiation.

Human breast carcinomas express high levels of the EGF receptors. Overexpression of this receptor has been associated with highly aggressive breast cancer types and a poor response to therapeutic agents. Prior preclinical and clinical studies have shown that blocking the EGFR via monoclonal antibodies or inhibition of EGFR tyrosine kinase with small molecule inhibitors inhibits the growth of breast cancers and sensitize chemotherapy responses. Single-chain antibodies to EGFR that contain the specific EGFR binding region but lack the Fc region have been isolated from human scFv phage display libraries. Yang et al., Small, 2009, 5(2):235-43, hereby incorporated by reference in its entirety, discuss the preparation of EGFR targeted nanoparticles conjugating a single-chain anti-EGFR antibody (ScFvEGFR).

Iron oxide nanoparticles conjugated to a purified antibody that selectively binds to the epidermal growth factor receptor (EGFR) deletion mutant (EGFRvIII) present on human glioblastoma multiforme (GBM) cells were used for therapeutic targeting and MRI contrast enhancement of experimental glioblastoma, both in vitro and in vivo, after convection-enhanced delivery (CED). See Hadjipanayis et al., Cancer Res, 2010, 70:6303, hereby incorporated by reference in its entirety. In certain embodiments, the disclosure relates to targeting moiety that is an antibody or antibody mimetic to EGFR or EGFRvIII for use in treating glioblastoma multiforme.

In certain embodiments, the targeting moiety is a monoclonal antibody-610 that targets a surface antigen for use in treating colon carcinoma. See Cerdan et al., Magn Reson Med, 1989, 12:151-63 1989, hereby incorporated by reference in its entirety.

In certain embodiments, the targeting moiety is an antibody to carcinoembryonic antigen (CEA) that targets CEA for use in treating colon tumors. See Tiefenauer et al., Magn Reson Imaging, 1996, 14:391-402, hereby incorporated by reference in its entirety.

In certain embodiments, the targeting moiety is a monoclonal antibody L6 that targets a surface antigen for use in treating intracranial tumor. See Remsen et al., Am J Neuroradiol, 1996, 17:411-18, hereby incorporated by reference in its entirety.

In certain embodiments, the targeting moiety is transferrin that targets transferrin receptor for use in treating carcinoma. See Kresse et al., Magn Reson Med, 1998, 40:236-42, hereby incorporated by reference in its entirety.

In certain embodiments, the targeting moiety is a monoclonal antibody to Her-2, e.g., Herceptin, that targets Her-2 receptors for use in treating breast cancer. See Lee et al., Nat Med, 2007, 13:95-9; Artemov et al., Magn Reson Med, 2003, 49:403-8; and Huh et al., J Am Chem Soc, 2005, 127:12387-91, all hereby incorporated by reference in their entirety.

In certain embodiments, the targeting moiety is the EPPT peptide that targets underglycosylated mucin-1 antigen (uMUC-1) for use in treating breast, colon, pancreas and lung cancer. See Moore et al., Cancer Res, 2004, 64:1821-7, hereby incorporated by reference in its entirety.

In certain embodiments, the targeting moiety is folic acid that targets folate receptor for use in treating mouth carcinoma and cervical cancer. See Chen et al., PDA J Pharm Sci Technol, 2007, 61:303-13; Sun et al., Small, 2006, 4:372-9; and Sonvico et al., Bioconjug Chem, 2005, 16:1181-8, all hereby incorporated by reference in their entirety.

In certain embodiments, the targeting moiety is methotrexate that targets folate receptor for use in treating cervical cancer. See Kohler et al., Langmuir, 2005, 21:8858-64, hereby incorporated by reference in its entirety.

In certain embodiments, the targeting moiety is a monoclonal antibody A7 that targets colorectal tumor antigen for use in treating colorectal carcinoma. See Toma et al., Br J Cancer, 2005, 93:131-6, hereby incorporated by reference in its entirety.

In certain embodiments, the targeting moiety is chlorotoxin peptide that targets membrane-bound matrixmetalloproteinase-2 (MMP-2) for use in treating glioma. See Veiseh et al., Nano Lett, 2005, 5:1003-8, hereby incorporated by reference in its entirety.

In certain embodiments, the targeting moiety is F3 peptide that targets surface-localized tumor vasculature for use in treating glioma. See Reddy et al., Clin Cancer Res, 2006, 12:6677-86, hereby incorporated by reference in its entirety.

In certain embodiments, the targeting moiety is RGD or RGD4C that targets integrins for use in treating melanoma and epidermoid carcinoma. See Zhang et al., Cancer Res, 2007, 67:1555-62 and Uchida et al., J Am Chem Soc, 2006, 128:16626-33, both hereby incorporated by reference in their entirety.

In certain embodiments, the targeting moiety is luteinizing hormone releasing hormone (LHRH) that targets LHRH receptor for use in treating breast cancer. See Leuschner et al., Breast Cancer Res Treat, 2006, 99:163-76, hereby incorporated by reference in its entirety.

In certain embodiments, the targeting moiety is CREKA peptide that targets clotted plasma proteins for use in treating breast cancer. See Simberg et al., Proc Natl Acad Sci U S A, 2007, 104:932-6, hereby incorporated by reference in its entirety.

In certain embodiments, the targeting moiety is an antibody to prostate specific membrane antigen (PSMA) that targets PSMA for use in treating prostate cancer. See Serda et al., Mol Imaging, 2007, 6:277-88, hereby incorporated by reference in its entirety.

### Lysosomally Degradation of Linkers

Macromolecules are typically taken up in cells by passive or active endocytosis. Endosomes are small vesicles that engulf macromolecules. They subsequently fuse with lysosomes containing a variety of enzymes effective in environment with a low pH. An “lysosomally degradable” linker or moiety refer to a chemical combination that degrades due to an enzyme present in lysosomes, or has accelerated degradation in a low pH, i.e., pH of less than 6. A typical lysosomally degradable linker is the polypeptide GFLG that is degradable by cathepsin B. Cathepsin B can cleave a number of protein sequences. See Peterson & Meares, Bioconjugate Chem., 1998, 9(5):618-626, hereby incorporated by reference. In certain embodiments, the lysosomally degradable linker comprises the sequence GXYZ (SEQ ID NO:2) wherein G is glycine, X is any amino acid, Y is valine, isoleucine, or leucine, and Z is alanine, glycine or glutamine. Alanine-phenylalanine and alanine-alanine sequences are degradable by cathepsin B. See Jeong et al., J Controlled Release, 2009, 137:25-30. In certain embodiments, linkers with alanine-phenylalanine and alanine-alanine sequences are contemplated. In certain embodiments, the term includes linkers comprising polyethylene glycols, esters, and acetals linked through, ester, amide or ether groups. See e.g., Kwon et al., Mol. Pharm., 2005, 2 (1): 83-91. In certain embodiments, linkers with urethane or urea groups or combinations thereof are contemplated. See Ouchi & Ohya, Prog. Polym. Sci., 1995, 20:211-257. In certain embodiments, linkers without ester groups are contemplated. In certain embodiments, the lysosomally degradable moiety is substantially stable to esterases or does not contain an ester.

### Antibodies, Fragments, Chimera, Antibody Mimetics, and Aptamers

In certain embodiments, the disclosure contemplates targeting moieties in any of the disclosed embodiments that are antibodies or fragments or chimera, antibody mimetics, or aptamers or any molecular entity that selectively binds receptors, proteins, or glycoproteins that are more prevalent on cancer cells.

Numerous methods known to those skilled in the art are available for obtaining antibodies or antigen-binding fragments thereof. For example, antibodies can be produced using recombinant DNA methods (U.S. Pat. No. 4,816,567). Monoclonal antibodies may also be produced by generation of hybridomas in accordance with known methods. Hybridomas formed in this manner are then screened using standard methods, such as enzyme-linked immunosorbent assay (ELISA) and surface plasmon resonance analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

The modular structure of antibodies makes it possible to remove constant domains in order to reduce size and still retain antigen binding specificity. Engineered antibody fragments allow one to create antibody libraries. A single-chain antibody (scFv) is an antibody fragment where the variable domains of the heavy (V_{H}) and light chains (V_{L}) are combined with a flexible polypeptide linker. The scFv and Fab fragments are both monovalent binders but they can be engineered into multivalent binders to gain avidity effects. One exemplary method of making antibodies and fragments includes screening protein expression libraries, e.g., phage or ribosome display libraries. Phage display is described, for example, in U.S. Pat. No. 5,223,409.

In addition to the use of display libraries, the specified antigen can be used to immunize a non-human animal, e.g., a rodent, e.g., a mouse, hamster, or rat. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. U.S. Pat. No. 7,064,244.

Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

In certain embodiments, a humanized antibody is optimized by the introduction of conservative substitutions, consensus sequence substitutions, germline substitutions and/or backmutations. An antibody or fragment thereof may also be modified by specific deletion of human T cell epitopes or “deimmunization” by the methods disclosed in U.S. Pat. No. 7,125,689 and U.S. Pat. No. 7,264,806. Briefly, the heavy and light chain variable domains of an antibody can be analyzed for peptides that bind to MHC Class II; these peptides represent potential T-cell epitopes. For detection of potential T-cell epitopes, a computer modeling approach termed “peptide threading” can be applied, and in addition a database of human MHC class II binding peptides can be searched for motifs present in the VH and VL sequences. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. The V BASE directory provides a comprehensive directory of human immunoglobulin variable region sequences. These sequences can be used as a source of human sequence, e.g., for framework regions and CDRs. Consensus human framework regions can also be used, e.g., as described in U.S. Pat. No. 6,300,064.

Antibody mimetics or engineered affinity proteins are polypeptide based targeting moieties that can specifically bind to targets but are not specifically derived from antibody V_{H} and V_{L} sequences. Typically, a protein motif is recognized to be conserved among a number of proteins. One can artificially create libraries of these polypeptides with amino acid diversity and screen them for binding to targets through phage, yeast, bacterial display systems, cell-free selections, and non-display systems. See Gronwall & Stahl, J Biotechnology, 2009, 140(3-4), 254-269, hereby incorporated by reference in its entirety. Antibody mimetics include affibody molecules, affilins, affitins, anticalins, avimers, darpins, fynomers, kunitz domain peptides, and monobodies.

Affibody molecules are based on a protein domain derived from staphylococcal protein A (SPA). SPA protein domain denoted Z consists of three α-helices forming a bundle structure and binds the Fc protion of human IgG1. A combinatorial library may be created by varying surface exposed residues involved in the native interaction with Fc. Affinity proteins can be isolated from the library by phage display selection technology. Affibody to HER-2 has been described. See Orlova et al., Cancer Res., 2007, 67:2178-2186, hereby incorporated by reference in its entirety.

Monobodies, sometimes referred to as adnectins, are antibody mimics based on the scaffold of the fibronectin type III domain (FN3). See Koide et al., Methods Mol. Biol. 2007, 352: 95-109, hereby incorporated by reference in its entirety. FN3 is a 10 kDa, β-sheet domain, that resembles the V_{H} domain of an antibody with three distinct CDR-like loops, but lack disulfide bonds. FN3 libraries with randomized loops have successfully generated binders via phage display (M13 gene 3, gene 8; T7), mRNA display, yeast display and yeast two-hybrid systems. See Bloom & Calabro, Drug Discovery Today, 2009, 14(19-20):949-955, hereby incorporated by reference in its entirety.

Anticalins, sometimes referred to as lipocalins, are a group of proteins characterized by a structurally conserved rigid β-barrel structure and four flexible loops. The variable loop structures form an entry to a ligand-binding cavity. Several libraries have been constructed based on natural human lipocalins, i.e., ApoD, NGAL, and Tlc. Anticalins have been generated for targeting the cytotoxic T-lymphocyte antigen-4 (CTLA-4) and the vascular endothelial growth factor (VEGF). See Skerra, FEBS J., 275 (2008), pp. 2677-2683, hereby incorporated by reference in its entirety.

The ankyrin repeat (AR) protein is composed repeat domains consisting of a β-turn followed by two α-helices. Natural ankyrin repeat proteins normally consist of four to six repeats. The ankyrin repeats form a basis for darpins (designed ankyrin repeat protein) which is a scaffold comprised of repeats of an artificial consensus ankyrin repeat domain. Combinatorial libraries have been created by randomizing residues in one repeat domain. Different numbers of the generated repeat modules can be connected together and flanked on each side by a capping repeat. The darpin libraries are typically denoted NxC, where N stands for the N-terminal capping unit, C stands for the C-terminal capping domain and x for the number of library repeat domains, typically between two to four. A HER-2 binding darpin has been generated from a library containing two randomized repeat domains (N2C library) and by an affinity maturation strategy. Zahnd et al., J. Mol. Biol., 2007, 369:1015-1028, hereby incorporated by reference in its entirety.

Aptamers refer to affinity binding molecules identified from random proteins or nucleic acids libraries. Peptide aptamers have been selected from random loop libraries displayed on TrxA. See Borghouts et al., Expert Opin. Biol. Ther., 2005, 5:783-797, hereby incorporated by reference in its entirety. SELEX (“Systematic Evolution of Ligands by Exponential Enrichment”) is a combinatorial chemistry technique for producing oligonucleotides of either single-stranded DNA or RNA that specifically bind to a target. Standard details on generating nucleic acid aptamers can be found in U.S. Pat. No. 5,475,096, and U.S. Pat. No. 5,270,163. The SELEX process provides a class of products which are referred to as nucleic acid ligands or aptamers, which has the property of binding specifically to a desired target compound or molecule. Each SELEX-identified nucleic acid ligand is a specific ligand of a given target compound or molecule. The SELEX process is based on the fact that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

### EXPERIMENTAL

### Preparation of Amino-Terminal Fragment-Iron Oxide Nanoparticles

A cDNA fragment encoding amino acids 1 to 135 of mouse or human uPA, isolated by PCR amplification using a PCR primer pair containing forward (5′-CACCATGGGCAGTGTACTTGGAGCTCC-3′) (SEQ ID NO:3) and reverse (5′-GCTAAGAGAGCAGTCA-3′) (SEQ ID NO:4) primers, was cloned into pET101/D-TOPO expression vector (Invitrogen). Recombinant amino-terminal fragment peptides were expressed in Escherichia coli BL21 (Invitrogen) and purified from bacterial extracts under native conditions using a Ni²⁺ nitrilotriacetic acid (NTA)-agarose column (Qiagen). Purification efficiency was determined by SDS-PAGE and >95% of purified proteins were amino-terminal fragment peptides.

Paramagnetic iron oxide nanoparticles were prepared using iron oxide powder as the iron precursor, oleic acid as the ligand, and octadecene as the solvent. Amino-terminal fragment peptides were conjugated to the surface of iron oxide nanoparticles via cross-linking of carboxyl groups to amino side groups on the amino-terminal fragment peptides. The polymer-coated iron oxide nanoparticles were activated with ethyl-3-dimethyl amino propyl carbodiimide (Pierce) and sulfo-N-hydroxysuccinimide for 15 min. After purification using Nanosep 100k OMEGA (Pall Corp.), activated iron oxide nanoparticles were reacted with amino-terminal fragment or dye-amino-terminal fragment peptides at a molar ratio iron oxide to amino-terminal fragment of 1:20 in PBS (pH 7.0) at 4° C. overnight, generating amino-terminal fragment-iron oxide or dye-amino-terminal fragment-iron oxide nanoparticles. The final amino-terminal fragment-iron oxide conjugates were purified using Nanosep 100k column filtration.

### Preparation and Characterization of Gem-IONPs and ATF-Gem-IONPs

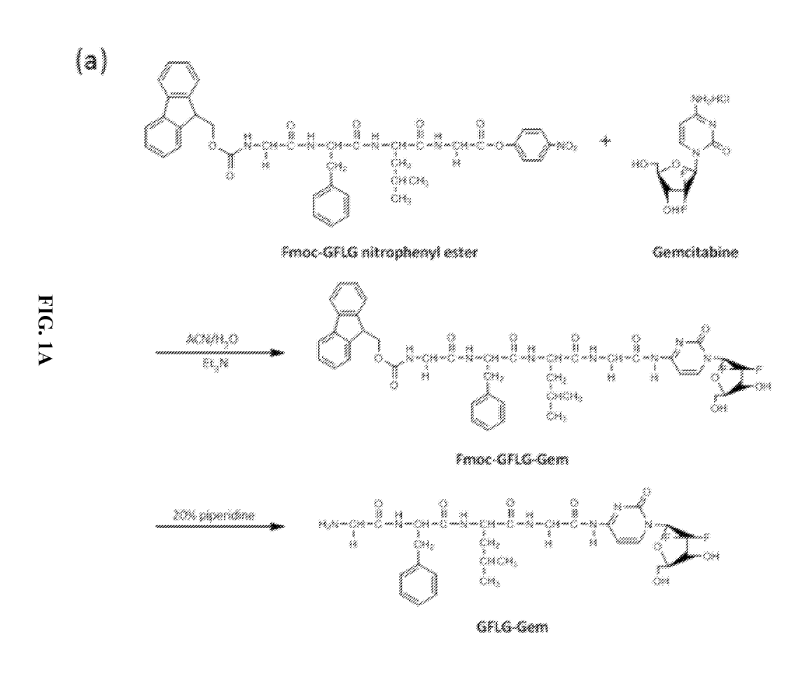

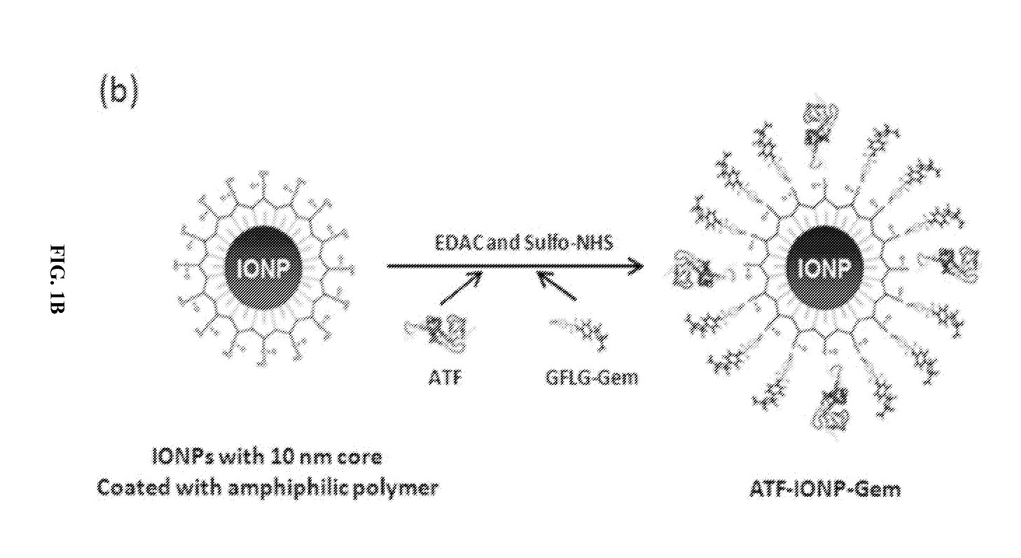

Magnetic IONPs were prepared using methods as described in Yu et al., Chem. Commun. (Camb) 2004, 20, 2306-2307, hereby incorporated by reference in its entirety. IONPs with uniform core sizes of 10 nm were utilized. Gemcitabine is a hydrophilic drug. It cannot be loaded in the hydrophobic core of the IONPs. Therefore, gemcitabine was attached on the surface of the IONPs via Gly-Phe-Leu-Gly (GFLG) (SEQ ID NO:1) tetra peptide linker that is lysosomally degradable (FIG. 1). Thus, gemcitabine can be released out from the IONPs after cleavage of GFLG spacer by lysosomal enzymes such as Cathepsin B. The purities of the purified recombinant mouse and human ATF peptides were confirmed by sodium dodecyl sulfate (SDS)—PAGE gel and the peptides appeared as monomers (17 kDa) or dimmers (35 kDa). The binding of ATF to uPAR receptor has species specificity. Human ATF peptides have a low binding affinity to mouse uPAR. Both human and murine recombinant ATF peptides were conjugated to the IONPs at the same weight ratio. The NPs adequately bind to human-derived MIA PaCa-2 cells and murine-derived blood vessels, respectively. Based on HPLC analyses, 570˜580 molecules of GFLG-Gem conjugates were bound to the surface polymer of IONPs and about 13 ATF peptides were linked to IONPs. After conjugation with GFLG-Gem conjugates and/or ATF peptides, the size of IONPs increased compared to 22 nm of original IONPs, as determined by dynamic light scattering. As shown in Table 1, Gem-IONPs and ATF-Gem-IONPs showed a size of 49.9 nm and 65.9 nm, respectively, which were gradually increased after conjugation but within the desired size range (10˜100 nm) for targeted drug delivery nanoparticle.

Conditional Release of Gemcitabine from ATF-Gem-IONPs

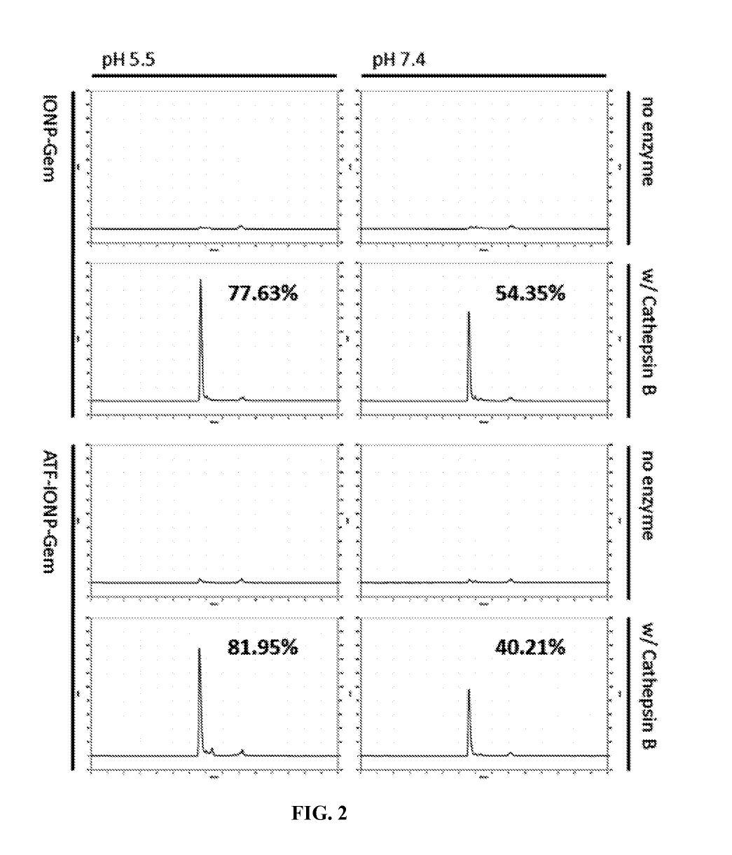

Whether gemcitabine is release out to kill pancreatic cancer cells was investigated. As shown in FIG. 2, gemcitabine peak was detected in the HPLC chromatograms only after incubation with Cathepsin B and released % of gemcitabine was 1.5 to 2 times-dominant at pH 5.5, than at pH 7.4. Incubation of the NPs at physiological pH enables 40% or 54% release of gemcitabine conjugated to Gem-IONPs or ATF-Gem-IONPs, respectively. At pH 5.5, 78% of gemcitabine conjugated to Gem-IONPs and 82% to ATF-Gem-IONPs had been released from the NPs. Conjugation of ATF peptides to IONPs did not affect the drug release efficiency (FIG. 2). Consequently, these results confirmed that gemcitabine can be easily released by lysosomal enzymes at lysosomal pH. Gemcitabine, which is linked to the IONPs through the GFLG linker peptide, can be conditionally released from Gem-IONPs or ATF-Gem-IONPs in the presence of cathepsin B, a lysosomal enzyme, under mild acidic conditions, which resembles pH of intracellular vesicles such as endosomes (pH 5.5˜6.0) and lysosomes (pH 4.5˜5.0) after 24 hr incubation.

### Targeting Activity of ATF-Gem-IONPs In Vitro

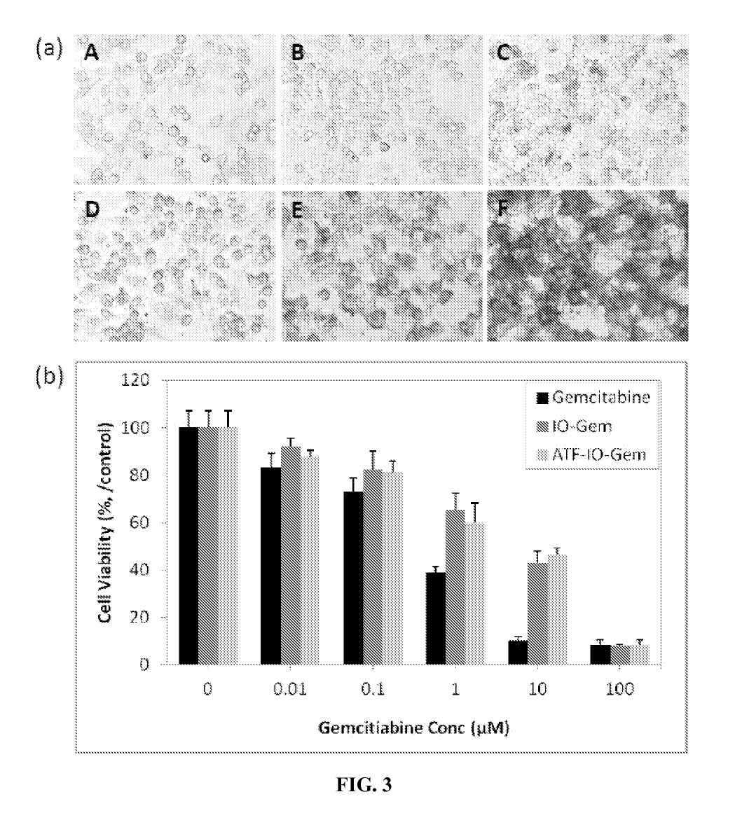

Whether ATF-Gem-IONPs specifically bind to the uPAR-expressing MIA PaCa-2 cells in vitro was evaluated to confirm the targeting activity of ATF peptide conjugated to the IONPs for effective drug delivery. After incubating cells with various IONPs at iron-equivalent concentration for 4 h, the iron amount bound to the cells was examined by Prussian blue staining method, given strong blue color produced by the stained iron. Incubation of human pancreatic cancer MIA PaCa-2 cells with uPAR-targeted ATF-Gem-IONPs resulted in the binding of NPs into the cells. IONPs nor non-targeted Gem-IONPs at 10 nM of iron-equivalent concentration resulted in binding (FIG. 3(a)). At 100 nM, a very low level of non-specific binding was present following incubation of the cells with Gem-IONPs, but a higher level of iron staining was observed in the cells treated with the uPAR targeted ATF-Gem-IONPs compared to the non-targeted Gem-IONPs. This confirms the specific binding of ATF-Gem-IONPs to uPAR-expressing human cancer cells and infers that the systemic delivery of ATF-Gem-IONPs leads to the target specific and effective delivery and accumulation of the NPs into the uPAR-expressing tumors in vivo.

### Cytotoxic Effect of ATF-Gem-IONPs on Pancreatic Cancer Cells

To evaluate the feasibility of uPAR-targeted ATF-Gem-IONPs for the treatment of pancreatic cancer, the anti-proliferative effect of free gemcitabine, non-targeted, or targeted NPs on MIA PaCa-2 cells were compared. For this experiment, the cells were incubated with free gemcitabine (Gem), Gem-IONPs or ATF-Gem-IONPs for 96 h, which may allow for uPAR-targeted IONPs to be internalized into the cells. Below 1 μM of gemcitabine-equivalent concentration, there was no significant difference between three groups. However, above 1 μM of gemcitabine-equivalent concentration, a remarkable inhibition of cell proliferation was found in free gemcitabine treated cells (FIG. 3(b)), while Gem-IONPs and ATF-Gem-IONPs did not effectively inhibit cell proliferation compared with gemcitabine. At the gemcitabine equivalent concentration of 1 μM, cell viability was about 65% for Gem-IONPs treated cells and 60% for ATF-Gem-IONPs, while the cell viability of gemcitabine treated cells decreased to 39%. And, IC₅₀ values increased from 0.498 μM for gemcitabine to 4.851 μM for Gem-IONPs and to 5.986 μM for ATF-Gem-IONPs. At the gemcitabine equivalent concentration of 100 the anti-proliferation effect of Gem-IONPs and ATF-Gem-IONPs finally reached to 8% of cell proliferation inhibition by gemcitabine.

### Inhibition of Pancreatic Tumor Growth by ATF-IO-Gem NPs

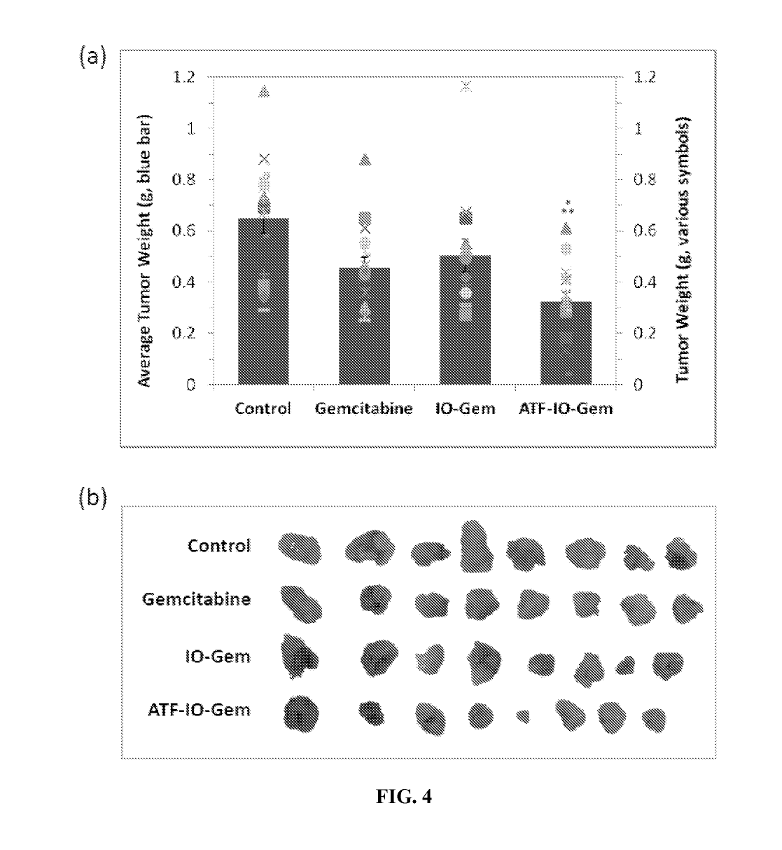

The tumor growth inhibition effect of ATF-Gem-IONPs was studied using female mice bearing orthotopical pancreatic cancer from an uPAR positive human pancreatic cancer cell line, MIA PaCa-2. NPs were administered five times at twice a week and the dose of ATF-Gem-IONPs was calculated based on gemcitabine-equivalence. Tumors were weighed at the end of the experiment, and average tumor weight was compared between groups (FIG. 4A). All of treated groups inhibited tumor growth compared to non-treated control group. Gemcitabine and non-targeted Gem-IONPs were found to inhibit tumor growth by 30% and 23%, respectively, but there were no statistically significant differences between both groups (p=0.4086). ATF-Gem-IONPs showed approximately 50% tumor growth compared to the untreated control and inhibited tumor growth about 34% and 28% versus free gemcitabine and non-targeted Gem-IONPs, respectively, and there were statistically significant difference. Tumor weights agreed well tumor volumes results (FIG. 4B). These results indicate that the uPAR-targeted ATF-Gem-IONPs have a high efficiency in delivery of drugs into tumor cells to produce efficient anti-tumor effect.

### Tumor Targeting Ability and MRI Contrast Effect of ATF-Gem-IONPs

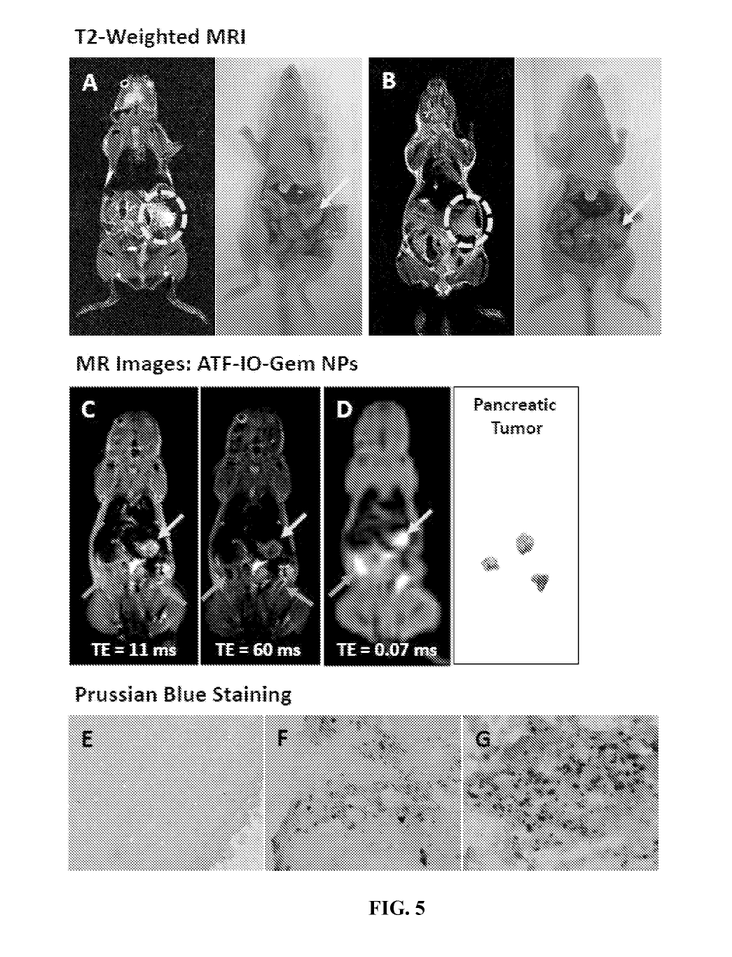

To monitor if the systemic delivery of ATF-Gem-IONPs leads to the target specific accumulation of the IONPs into the uPAR-expressing tumors in vivo and to investigate efficiency and treatment response of our theranostic NPs as a drug delivery system for pancreatic cancer therapy, the animals were scanned with MRI at 48 h after final injection. Through MRI analysis, tumor size was observed and compared between groups before dissection. When comparing the T2-weighted MR images of the tumor-bearing mice treated with Gem-IONPs or ATF-Gem-IONPs, remarkable signal decrease was detected in the tumor area following repeated injection of the ATF-Gem-IONPs compared to injection of non-targeted Gem-IONPs, indicating selective accumulation of the NPs in the pancreatic cancers (FIG. 5A). Results of the T2-weighted fast spin echo MRI showed that the ATF-Gem-IONPs selectively accumulated within the pancreatic tumors, as evidenced by a decrease in MRI signal in the area of the tumors, and Ultrashort echo time (UTE) MRI of the same mice showed reversely significant bright UTE signal in the multiple tumor lesions (FIG. 5B). However, the pancreatic tumors in mice that received non-targeted Gem-IONPs did not have detectable MRI signal changes. In addition, UTE MRI of the mice that received Gem-IONPs were also performed and a significant bright UTE signal in the tumor was not detected. To estimate the level of IONPs in the tumor tissues, the MRI signal changes were measured in the representative areas of the tumor in the mice that received either targeted or non-targeted Gem-IONPs. Using the MRI signal level of muscle tissue as a baseline, there is a 4.8 fold change in the MRI signal within the pancreatic tumor of the mouse treated with ATF-Gem-IONPs compared to the mouse that received non-targeted IO NPs. In addition, to confirm the accumulation of non-targeted Gem-IONPs or ATF-Gem-IONPs in tumor tissues, Prussian blue staining was performed on the frozen tissue sections harvested at 48 h after the injection of NPs. Prussian blue stained cells were found in the tumor sections from the mice treated with ATF-Gem-IONPs but not in the tissue sections of the mice treated with non-targeted Gem-IONPs (FIG. 5C). Examination of the microscope images under high magnification showed the intracellular localization of the Prussian blue iron stain within the cells. Taken together, these results indicate that ATF-Gem-IONPs selectively accumulate in pancreatic cancers after systemic delivery and that NPs deliver gemcitabine effectively to tumor site and thus enhance the tumor inhibition effect and signal of MRI.

### Synthesize Bio-Reactive Near Infrared Dyes for In Vivo Optical Imaging

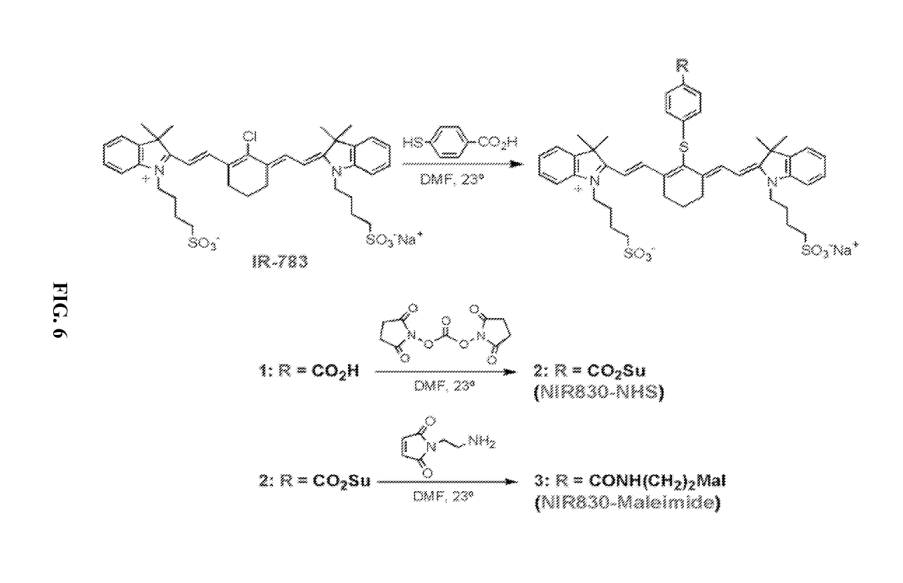

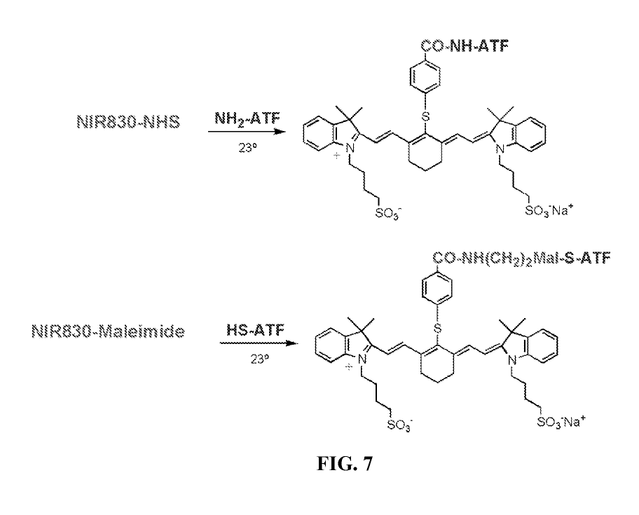

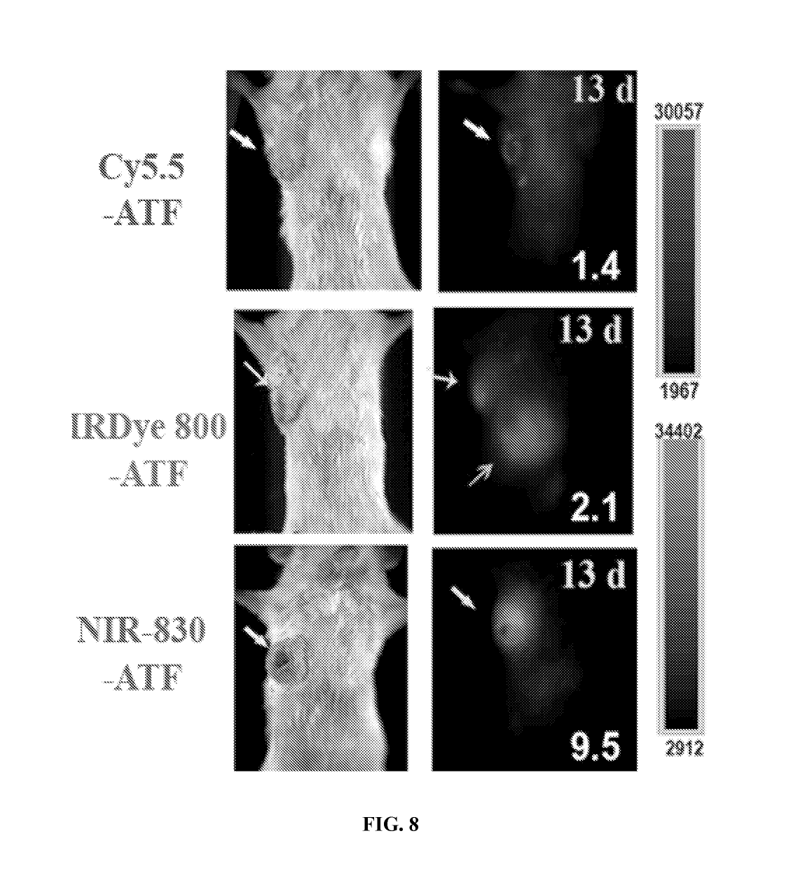

NIR dyes (NIR 830-NHS and NIR830-Maleimide) were prepared that contain amine reactive succinimidyl (NHS) ester (NIR830-NHS) and thiol-reactive maleimide functional groups (NIR830-Maleimide) for conjugation to peptides, proteins or other molecules. See FIG. 6. NIR830-NHS is used for direct conjugation with an amino functionality of peptides or proteins by forming covalent peptide bond. The double bond in NIR830-Maleimide readily reacts with the thiol group found on peptides or proteins to form a stable carbon-sulfur bond. See FIG. 7. These dyes are stable for over 12 month in vitro and 5 weeks in vivo. Conjugation of the dye molecules to peptide- or antibody-based ligands results in NIR optical imaging probes that can be excited at 625 to 800 nm and emit at 825 nM wavelengths.

Imaging of Cancer with NIR-830

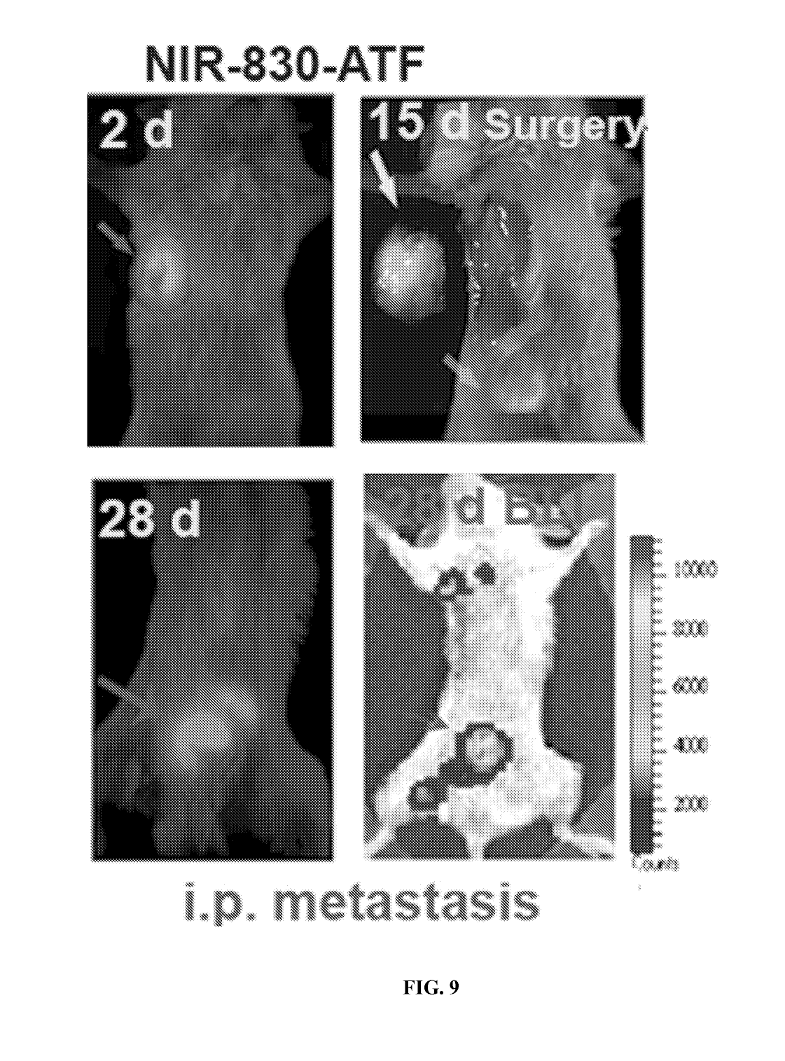

Systemic delivery of NIR-830 dye labeled and targeted optical imaging probes allows for specific imaging of primary and metastatic cancer lesions in several orthotopic breast, pancreatic, colon, head and neck, and ovarian cancer animal models. NIR-830, that can be excited at wavelengths of 725 to 800 nm and an emission (Em) peak at 825 nm for peptide-conjugates. Production of optical imaging probes for in vivo imaging requires the use of a NIR dye that generates a strong fluorescence and high signal/background ratio. NIR-830, that can be excited at wavelengths of 725 to 800 nm and an emission (Em) peak at 825 nm for peptide-conjugates. In vivo optical imaging results in 4T1 mouse mammary tumor model showed that among three NIR dye-labeled ATF peptides that are administrated at the same amount, NIR 830-ATF had the strongest signal in the tumor and the lowest background in the liver and body compared to two commercial dyes. The growth of the primary tumor and metastatic lesions can be monitored using optical imaging (FIG. 9).

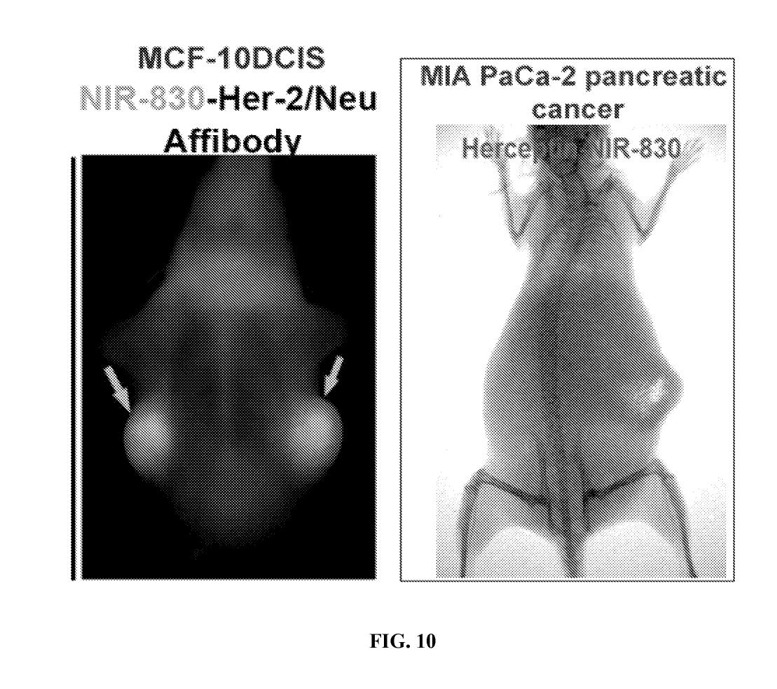

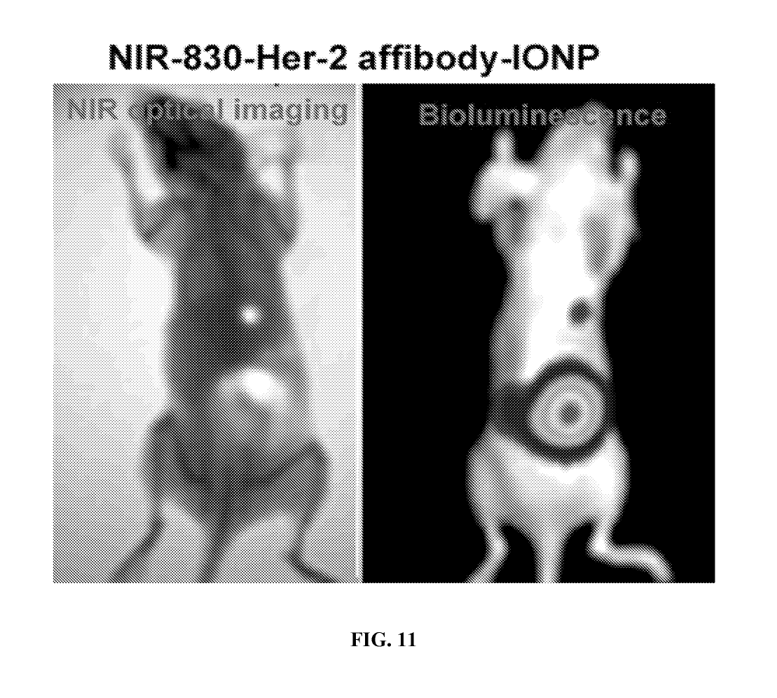

For image-guided surgery, the most important property is that the NIR-830-dye stays in the tumor, but not in normal organs, for over four weeks after being internalized into tumor cells. Among three dye-labeled optical imaging probes, only NIR830 dye imaging probe retains NIR signal in the tumor for over 4 weeks and the imaging signal increases as the growth of the tumor mass. Therefore, NIR830 dye-labeled targeted imaging probes can be used for intraoperative detection of tumor margin, in vivo imaging of primary and metastatic cancer lesions as well as monitoring the changes in tumor size. NIR-830 dye can be labeled to other forms of targeting ligands, such as affibody or monoclonal antibody (Herceptin) to detect Her-2 positive tumors. See FIGS. 10 and 11.

Targeted Nanoparticles Carrying Therapeutic Agents within Hydrophobic Polymer Covering the IOPN

Given that coating co-polymers are made of hydrophobic and hydrophilic layers containing functional groups, therapeutic agents can be introduced to the nanoparticles via several different approaches, depending on the chemical properties and release mechanisms of the drugs. While hydrophobic drug can be encapsulated in the hydrophobic block of the co-polymer before or during coating IONP, hydrophilic drug can be conjugated to the surface functional groups using an enzyme cleavable linker.

Several hydrophobic drug molecules can be efficiently encapsulated into the hydrophobic space between the iron core of the nanoparticles and amphiphilic polymer coating. The following drug molecules can be loaded onto the nanoparticles at ratios from 0.6 to 1 mg of drug to 1 mg of Fe (or 900 pmol of IONPs): Doxorubicin, ABT888, Erlotinib, docetaxel and paclitaxel. Of these drugs, doxorubicin and ABT888 also show pH-dependent drug release since they have an amine side group that is protonated under low pH conditions (ph 4.0 to 5.0), which convert the drug molecules to hydrophilic molecules and release from the polymer.

One protocol for encapsulation of hydrophobic drugs into amphiphilic polymer coated magnetic iron oxide nanoparticles is as follows. Drug, e.g., 10 mg, is dissolved in 200 μl of methanol. The drug solution is slowly added to 900 pmol of nanoparticles (or 1 mg of iron oxide) in 1 ml water (pH 8.5). The ratio of the drug:IONP is depending on chemical properties of drug molecules and surface modifications of the nanoparticles. The mixture is slowly stirred for 4 hrs in room temperature. Drug-nanoparticle conjugates and free drug molecules can be separated by the following two methods: 1). place in a magnet-sorter (Ocean Nanotech, LLC) for overnight in 40 C and then remove supernatant; or 2). Centrifuge using a Nanosep 100k OMEGA filter column (Pall Corp, Ann Arbor, Mich.) at 6000 rpm for 10 min.

### Dual Imaging Modality Theranostic Iron Oxide Nanoparticles (IONPs)

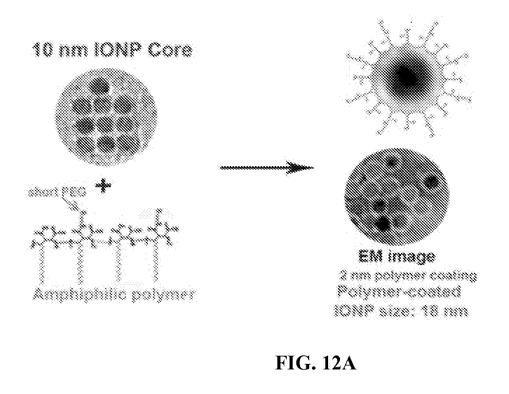

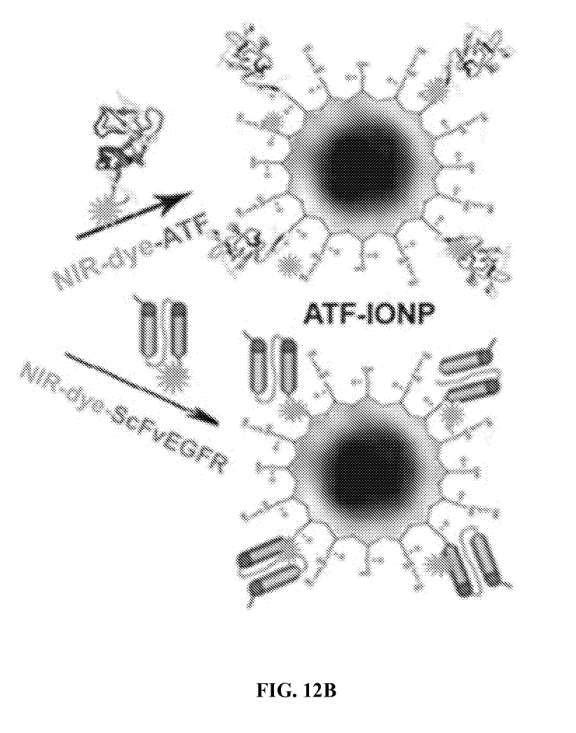

IONPs were functionalized with an amphiphilic copolymer layer, which stabilizes the surface of the IONPs and forms a size compact IONP with more than 400 active carboxyl groups that are readily available for conjugation with targeting ligands and therapeutic agents (FIG. 12A).

Targeted theranostic IONPs comprises targeting ligands desirably with the following properties:
1) high affinity for a cell surface receptor that is highly expressed in tumors; 2) a low toxicity and immunogenicity; and 3) a small molecular size and capability for mass production. Two recombinant peptides have been produced from bacterial expressing systems with these features.
Amino terminal fragment (ATF, 135 aa) of mouse or human uPA (17 KDa) has a high binding affinity to uPAR (K_{d}<1 nM), and can compete with uPA binding to uPAR and block uPAR function. This enables ATF-IONPs to bind to uPAR expressing cells in the presence of a high level of endogenous uPA. A single chain antibody to EGFR (ScFvEGFR, 25 KDa, K_{d}=3.4 nM) is a human antibody that reacts with both human and mouse.

To produce targeted optical and MR imaging probes, ATF or ScFvEGFR peptides were labeled with a new NIR dye (NIR-830) and then conjugated to the amphiphilic polymer coated IONPs (FIG. 12D). About 12 to 16 peptides and 48 to 64 dye molecules were conjugated to one IONP.

Since coated co-polymers are comprised of hydrophobic and hydrophilic layers containing functional groups, drugs can be introduced into the IONPs via various approaches, depending on their chemical properties and release mechanisms. Hydrophobic doxorubicin (Dox), ABT-888, erlotinib or taxol can be encapsulated in the hydrophobic inner layer of the IONPs via hydrophobic interactions (FIG. 12). Both Dox and ABT-888 can be efficiently incorporated into the amphiphilic polymer coated IONPs by mixing the IONPs with appropriate ratio of Dox or ABT-888 at pH 8.0 for 4 hours. The drug loading efficiency is about 15% (w/w) of the IONP, which is comparable to that of many polymeric nanoparticles. Cisplatin and gemicitabine have been conjugated onto the surface of the nanoparticles. Cisplatin was conjugated to nanoparticle surface through ion-complex formation between cis-platinum (II) and carboxyl group of the polymer coating.

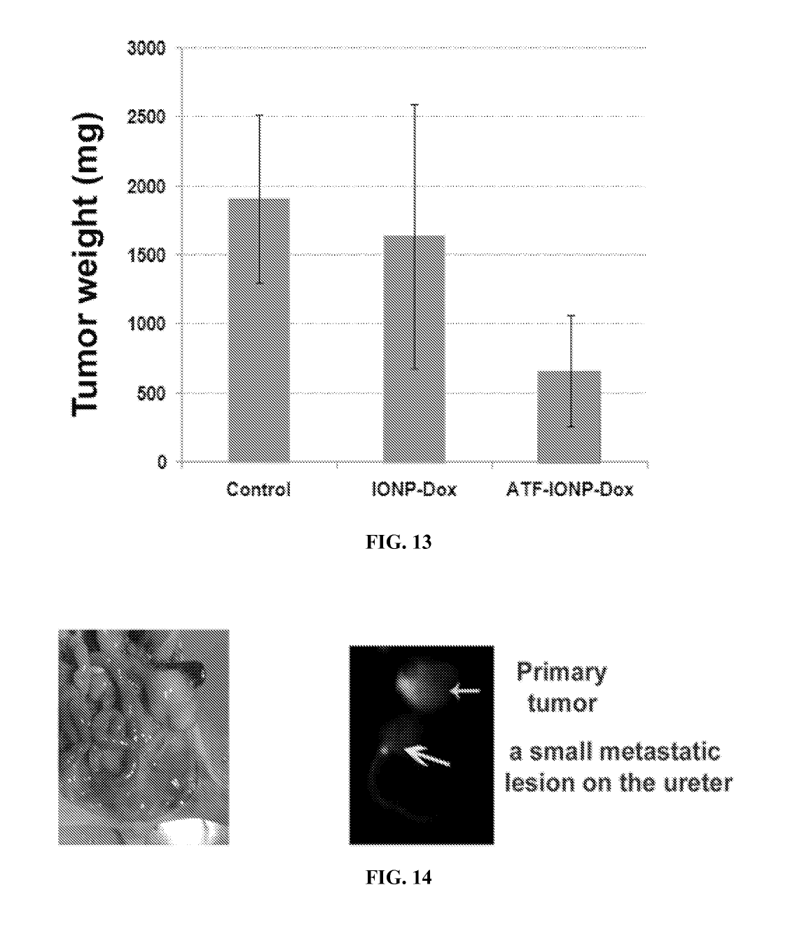

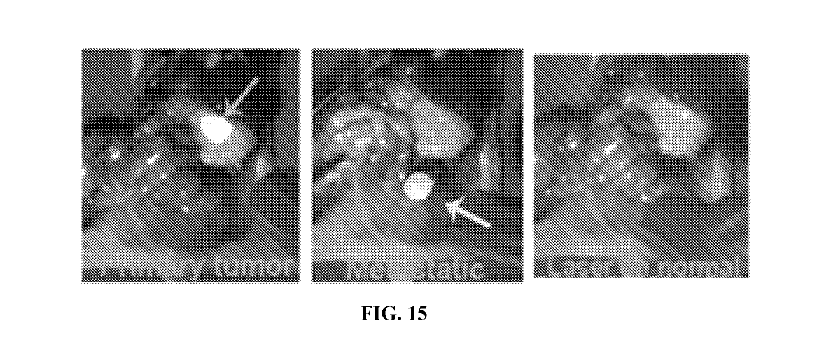

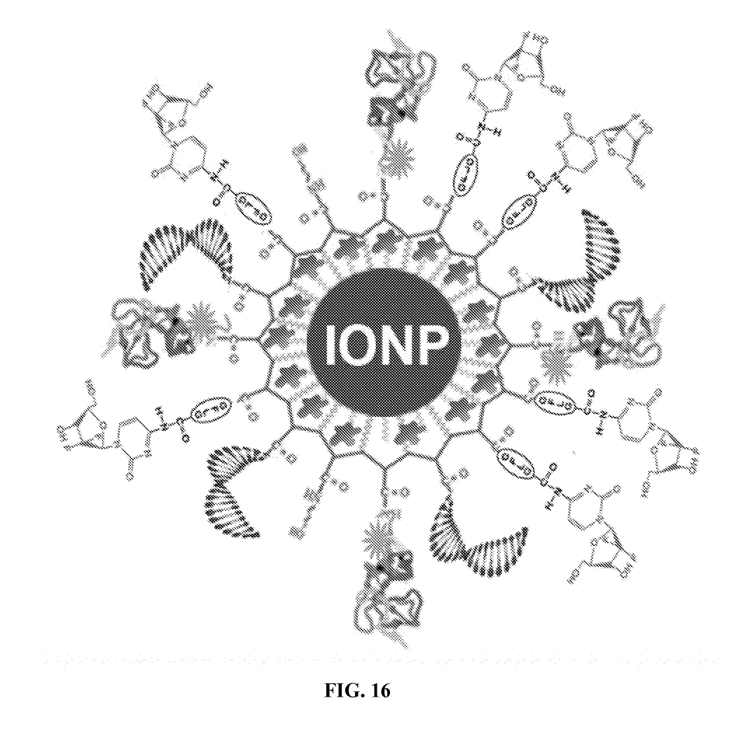

Dual imaging theranostic IONP-Dox has been executed in an orthotopic human pancreatic cancer tumor xenograft model in nude mice. Following systemic delivery of four doses of NIR-830-ATF-IONP-Dox at 10 mg/Kg Dox concentration, MRI can detect changes in tumor size and contrast. The total tumor weight is significantly decreased in the nude mice treated with NIR-830-dye-ATF-IONP-Dox (FIG. 13). The ability of Kodak in vivo optical system and SpectroPen in detecting residual drug resistant primary tumor and a small peritoneal metastatic lesion, which could not be detect by visualization, is illustrated in FIGS. 14 and 15. Histological analysis revealed that a high level of ATF-IONP-Dox can be internalized by tumor cells in the invasive tumor burdens, invaded lymph nodes, peritoneal metastasis and lung micrometastasis. Those cells are also positive for CD44 and CD24, which are markers for pancreatic cancer stem cells.

1. A particle comprising a core coated with a polymer, wherein the polymer is conjugated to a targeting moiety, lysosomally degradable moiety, and a therapeutic agent.

2. The particle of claim 1, wherein the therapeutic agent is gemcitabine, doxorubicin, cytosine arabinoside, mitomycin, cisplatin, or any therapeutic agent with that an amine side group.

3. The particle of claim 1, wherein particle is an iron oxide nanoparticle or quantum dot.

4. The particle of claim 1, wherein the particle has a diameter of between about 10 and 200 nm.

5. The particle of claim 1 wherein the lysosomally degradable moiety is the polypeptide GFLG (SEQ ID NO:1).

6. The particle of claim 1 wherein the lysosomally degradable moiety is linked to the therapeutic agent.

7. The particle of claim 1, where in the lysosomally degradable moiety linked to the therapeutic agent is of the formula: 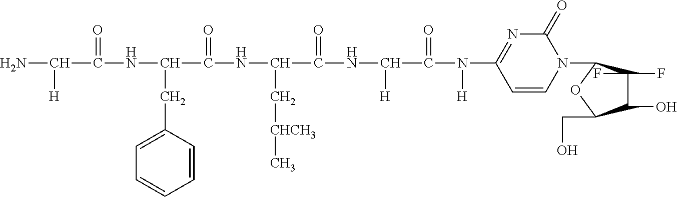
or salts thereof optionally substituted with one or more substituents.

8. The particle of claim 1, wherein the polymer is an amphiphilic polymer.

9. The particle of claim 1, wherein the polymer comprises a hydrophobic section further comprising a hydrophobic therapeutic agent.

10. The particle of claim 1, wherein the targeting moiety binds uPAR, EGFR, or HER-2.

11. The particle of claim 10, wherein the targeting moiety is an HER-2 or EGFR antibody or affibody, uPA, or fragment thereof.

12. The particle of claim 1, wherein the targeting moiety is the amino-terminal fragment of uPA or single chain antibody to EGFR.

13. The particle of claim 1 wherein the particle further comprises a (3,3-dimethyl-indol-1-ium-1-yl)-N-alkylsulfonate dye or salt thereof.

14. The particle of claim 13, wherein the dye has the formula: 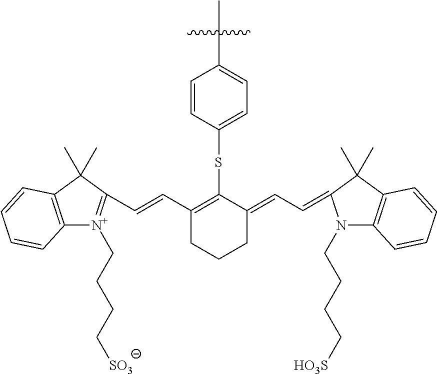
or salts thereof optionally substituted with one or more substituents.

15. A pharmaceutical composition comprising a particle of claim 1 and a pharmaceutically acceptable excipient.

16. A method of treating or preventing a disease or condition comprising administering an effective amount of a pharmaceutical composition of claim 15 to a subject in need thereof.

17. The method of claim 16, wherein the disease is a cancer that forms tumors.

18. The method of claim 17, wherein the cancer overexpresses a receptor of the targeting molecule in tumor cells, tumor endothelial cells, or tumor stromal fibroblasts compared to noncancerous tissue of an organ containing the cancerous tumor.

19. The method of claim 17, wherein the cancer overexpress uPAR, EGFR, or HER-2.

20. The method of claim 17, wherein the cancer is selected from pancreatic cancer, breast cancer, prostate cancer, lung cancer, skin cancer, bladder cancer, brain cancer, colon cancer, rectal cancer, kidney cancer, endometrial cancer, and thyroid cancer.


SEQUENCE LISTING (4 sequences)

SEQ ID NO: 1 - protein, 4 aa, Artificial Sequence
  Polypeptide
GFLG

SEQ ID NO: 2 - protein, 4 aa, Artificial Sequence
  lysosomally degradable linker
GXYZ

SEQ ID NO: 3 - DNA, 27 bp, Homo sapiens
caccatgggcagtgtacttggagctcc

SEQ ID NO: 4 - DNA, 16 bp, Homo sapiens
gctaagagagcagtca